(19) United States
(12) Patent Application Publication
(10) Pub. No.: US 2004/0241219 A1
(43) Pub. Date: Dec. 2, 2004

(54) Transdermal therapeutic system based on polyacrylate-contact-bonding adhesives without functional groups

(76) Inventors: Thomas Hille, Neuwied (DE); Frank Theobald, Bad Breisig (DE); Ursula Hildegard Klein, Neuwied (DE)

Correspondence Address: William F Lawrence, Frommer Lawrence & Haug, 745 Fifth Avenue, New York, NY, 10151, US

(21) Appl. No.: 10/487,380

(22) PCT Filed: Aug. 13, 2002
(86) PCT No.: PCT/EP02/09057

(30) Foreign Application Priority Data
Aug. 24, 2001 (DE) 101416520

(51) Int. Cl. A61K 9/70

(52) U.S. Cl. 424/449

(57) ABSTRACT

The invention relates to a transdermal therapeutic system (TTS) consisting of a rear layer, a protective layer and an active-substance-containing polymer layer. The polymer matrix comprises a polyacrylate which contains an extremely reduced number of functional groups. In one particular embodiment, the polyacrylate is free from hydroxyl groups and/or carboxyl groups.

Transdermal therapeutic systems (TTS) are flat pharmaceutical products built up in layers, in which one or more active compounds are embedded in an optionally contact-adhesive polymer matrix with or without excipients (e.g. penetration accelerators). As a rule, this polymer matrix is prepared by coating a support film with the polymer material containing the active compound and then providing it with a covering film, which also remains on the skin during the application of the transdermal therapeutic system. The support film serves as a protective layer for the polymer matrix during the storage period and optionally as an application aid for the later application of the transdermal therapeutic system.

Transdermal therapeutic systems make possible a continuous supply of active compound over the entire application period. They are therefore comparable with continuous drip infusions with respect to their concentration-time profiles. Numerous transdermal therapeutic systems containing different active compounds and active compound combinations are found today on the pharmaceutical market. One of the most important indication areas for transdermal therapeutic systems is hormone substitution therapy, in particular in the case of women in the menopause. In the early years of transdermal hormone substitution therapy, estrogen-containing monopreparations were especially employed therefor. Recently, however, transdermal therapeutic systems are being supplied which contain a combination of estrogens (e.g. 17β-estradiol) and gestagens (e.g. norethisterone). Testosterone, the male sex hormone, likewise belongs to the group consisting of the steroid hormones, which are used in the course of hormone substitution therapy, in particular in the treatment of hypogonadism.

A number of commercially obtainable transdermal therapeutic systems are constructed as “matrix systems”. These are systems in which the polymer matrix, which is equipped to be contact-adhesive or non-contact-adhesive, contains the active compound in dissolved or suspended form. The polymer matrix in this case usually consists of contact adhesives based on polyacrylates.

The polyacrylates used in this case are prepared from monomers (acrylic acid and methacrylic acid, and in each case their esters, optionally with vinyl acetate), which contain functional groups. These functional groups can survive the polymerization of the monomers employed unchanged and influence the properties of the resulting polyacrylate—in particular the tackiness and the adhesive power—crucially.

Thus, adhesive formulations based on polyacrylate are disclosed in EP 614 356, in which the content of the total of acrylic acid, glycidyl methacrylate and hydroxyethyl acrylate is between 4.8 and 5.5% by weight (cf. table 3 of this document).

The person skilled in the art distinguishes polyacrylates having —OH groups (hydroxyl groups) and those having —COOH groups (carboxyl groups) as functional groups. The polyacrylates containing hydroxyl groups include, for example, Durotak 2287, the polyacrylates containing carboxyl groups, for example, Durotak 2051, which are both produced by National Starch. These polyacrylates have proven to be stable and highly tolerable contact-adhesive polymers for the production of transdermal therapeutic systems which contain steroid hormones as active compounds.

A disadvantage in the case of the transdermal therapeutic systems whose polymer matrices contain polyacrylates which contain the functional groups mentioned (hydroxyl group, carboxyl group) is the low active compound utilization. This is to be observed in particular in hormone-containing transdermal therapeutic systems. In this case, a low active compound utilization is to be understood as meaning that, after expiry of the intended administration period of the transdermal therapeutic system, a relatively large amount of the active compound remains unutilized in the “used” transdermal therapeutic system in comparison with the total amount of the active compound contained therein before the start of the administration of this transdermal therapeutic system.

Since in some cases very expensive active compounds are employed in transdermal therapeutic systems, the low active compound utilization is undesirable from economic and from ecological points of view. Finally, in the case of pharmaceutical active compounds having a toxic action in relatively high concentration, a high residual content can also constitute a certain risk potential for the improper taking of a higher dose.

According to the invention, this disadvantage is solved by means of a transdermal therapeutic system which, as the base polymer for the polymer matrix, contains a contact-adhesive polyacrylate which contains an extremely reduced content of hydroxyl groups and/or carboxyl groups, so that this can be described as “essentially free of functional groups”. This is all the more surprising, since among experts the presence of functional groups, in particular hydroxyl groups and/or carboxyl groups, in the polyacrylates is considered as a prerequisite for a good cohesion and/or adhesion of the polymer matrix.

Suitable polyacrylates which according to the invention are “essentially free of functional groups” are polymers (homopolymers, copolymers and block copolymers) based on acrylic acid esters and/or methacrylic acid esters.

Suitable monomers for the preparation of the polyacrylate according to the invention are in this case in particular n-butyl acrylate, n-butyl meth-acrylate, ethyl acrylate, 2-ethylhexyl acrylate, ethyl methacrylate, methyl acrylate, methyl methacrylate, tert-butyl acrylate, sec-butyl acrylate, tert-butyl methacrylate, cyclohexyl methacrylate, 2-ethylhexyl methacrylate, isobornyl methacrylate, isobutyl methacrylate, isopropyl acrylate, isopropyl methacrylate and mixtures of these monomers. These monomers are esters of acrylic and methacrylic acid which contain linear, branched or cyclic aliphatic C₁-C₁₂ substituents without other functional groups.

Vinyl acetate can also be used as a comonomer for the preparation of the polyacrylate together with at least one of these monomers. The content of vinyl acetate in the monomer mixture used for the preparation of the polyacrylate should be below 20% by weight, preferably below 5% by weight. A vinyl acetate content of below 1.5% by weight is particularly preferred.

The esters of acrylic acid or methacrylic acid which carry functional groups and can be contained in the monomer mixture used for the preparation of the polyacrylate are primarily to be understood as meaning esters containing hydroxyl groups, that is 2-hydroxy-ethyl acrylate, 2-hydroxyethyl methacrylate, 3-hydroxy-propyl acrylate and 3-hydroxypropyl methacrylate. However, substances such as acrylonitrile, methacrylonitrile, acrylamide, dimethylaminoethyl acrylate etc can also be be considered within the meaning of this description as “esters of acrylic acid or methacrylic acid containing functional groups”.

However, the proportion of the total of acrylic acid, methacrylic acid, 2-hydroxyethyl acrylate, 2-hydroxyethyl methacrylate, 3-hydroxypropyl acrylate and/or 3-hydroxypropyl methacrylate in the monomer mixture used for the preparation of the polyacrylate is below 2% by weight, preferably below 1.5% by weight and particularly preferably below 0.2% by weight.

“Essentially free of functional groups” within the meaning of the present description is thus to be understood as meaning that the total content of acrylic acid, methacrylic acid and esters of acrylic acid or methacrylic acid which carry functional groups (in particular the esters containing hydroxyl groups) in the polyacrylate is below 2% by weight, preferably below 1.5% by weight. In a particular embodiment, the total content of these monomers is below 0.2% by weight. In a particular embodiment, none of these esters of acrylic acid or methacrylic acid which carry functional groups are contained in the monomer mixture.

The monomer mixtures can be polymerized in various ways, e.g. by ionic, free-radical or light-induced means etc., optionally using crosslinkers such as, for example, aluminum acetylacetonate, allyl glycidyl ether and/or glycidyl methacrylate (which—if present—are contained in the monomer mixture in a content of below 0.5% by weight) and optionally also using auxiliaries such as antioxidants, stabilizers and/or alkyl-mercaptans (which—if present—are contained in the monomer mixture in a content of below 0.1% by weight). Water, optionally together with emulsifiers or organic solvents, can be used as the reaction medium.

Contact adhesives based on polyacrylates, which in our view come under this definition of “essentially free of functional groups” are the Elite adhesives from National Starch and GMS 3083 from Solutia.

In a particularly simple embodiment, the polymer matrix consists exclusively of the active compound (or an active compound combination) and the polyacrylate according to the invention. However, embodiments are also possible in which a mixture of a polyacrylate without a functional group is used with a polyacrylate containing functional groups.

Beside the preferred hormones, in particular the steroid hormones, other pharmaceutically active substances can also be used as active compounds in the polymer matrices based on polyacrylates without functional groups. The following substances are suitable for this:

α-adrenoreceptor agonists such as, for example, xylometazoline, adrenolone, clonidine, ephedrine, tiamenidine,

β-adrenoreceptor agonists such as, for example, formoterol, terbuterol, ritodrine,

α-adrenoreceptor blockers such as, for example, dapiperazole, doxazosine, prazosine, yohimbine, trimazosine,

β-adrenoreceptor blockers such as, for example, acebutolol, atenolol, bisoprolol, bopindolol, bupranolol, propanolol, metoprolol, nadolol, pindolol, timolol,

anabolics such as, for example, androstenediol, bolandiol, clostebol, 4-hydroxy-19-nortestosterone, methenolone,

analgesics (narcotics) such as, for example, alfentanil, buprenorphine, codeine, dimenoxadol, fentanyl, isomethadone, lofentanil, methadone, morphine, morphine derivatives, normethadone, normorphine, propiram, sufentanil, tilidine,

analgesics (non-narcotics) such as, for example, aminopyrine, antipyrine, aspirin, benoxaprofen, bucetin, clometacin, etodolac, felbinac, fenoprofen, flubiprofen, ibufenac, indomethacin, indoprofen, ketoprofen, keterolac, miroprofen,

androgens such as, for example, boldenone, fluoxymesterone, mestanolone, mesterolone, methandrostenolone, 17-methyltestosterone, 17α-methyltestosterone 3-cyclopentyl enol ether, norethandrolone, normethandrone, oxandrolone, oxymetholone, prasterone, stanolone, stanozolol, testosterone, testosterone 17-chloral hemiacetal, testosterone 17β-cypionate, testosterone enanthate, testosterone nicotinate, testosterone phenylacetate, testosterone propionate, tiomesterones,

anesthetics such as, for example, amucaine, amylocalne, biphenamine, cocaine, diperodone, ecgonidine, euprocine, fenalcomine, fomocaine, hexobarbital, hexylcaine, hydroxydione, hydroxyprocaine, hydroxytetracaine, ketamine, leucinocaine mesylate, levoxadrol, lidocaine, mepivacaine, meprylcaine, metabutoxycaine, methohexital, midazolam, naepaine, octacaine, orthocaine, oxethazaine, parethoxycaine, phenacaine, piperocaine, polidocanol, pramoxine, prilocalne, procaine, propanocaine, propofol, risocaine, tetracaine, thialbarbital, thiamylal, thiobutabarbital, thiopental, tolycaine, trimecaine, zolamine,

antiallergics such as, for example, amlexanox, astemizole, azelastine, cromolyn, fenpipran, histamine, repirinast, tiaramide, tranilast, traxanox, urushiol, ketotifen, nedocromil, oxatomide, pentigetide,

antiandrogens such as, for example, bifluranol, cyoctol, cyproterone, oxendrolone,

antianginals such as, for example, amlodipine, amyl nitrite, cinepazet maleate, imolamine, isosorbide dinitrate, limaprost, molsidomine, nitroxyalkylamide derivatives,

antiarrhythmics such as, for example, acecainide, adenosine, ajmaline, alprenolol, amoproxan, aprindine, bretylium tosylate, bubumolol, bunaftine, butidrine, butobendine, meobentine, mexiletine, moricizine, pirmenol, pronethalol, propafenone, pyrinoline,

penicillins such as, for example, amdinocillin, pivoxil, amoxicillin, ampicillin, apalcillin, aspoxicillin, azidocillin, azlocillin, bacampicillin, benzylpenicillin, carbenicillin, carfecillin, carindacillin, clometocillin, cloxacillin, cyclacillin, dicloxacillin, diphenicillin, epicillin, fenbenicillin, floxicillin, hetacillin, lenampicillin, metampicillin, methicillin, mezlocillin, nafcillin, oxacillin, penamecillin, penethamate hydriodide, penicillin G benethamine, penicillin G benzathine, penicillin G benzhydrylamine, penicillin G calcium, penicillin G hydrabamine, penicillin N, penicillin O, penicillin V, penicillin V benzathine, penicillin V hydrabamine, penimepicycline, phenethicillin, piperacillin, pivapicillin, propicillin, quinacillin, sulbenicillin, talampicillin, temocillin, tiacarcillin,

antidiabetics such as, for example, sulfonylurea derivatives, acetohexamide, carbutamide, chlorpropamide, glibornuride, gliclazide, glimepiride, glipizide, gliquidone, glisoxepide, glyburide, glybuthiazole, glybuzole, glyhexamide, glymidine, glypinamide, phenbutamide, tolazamide, tolbutamide, tolcyclamide, acarbose, benzylthiazolidine-2,4-dione, calcium mesoxalate, miglitol,

antihistaminics such as, for example, acrivastine, bamipine, brompheniramine, chlorpheniramine, dimethindene, metron S, pheniramine, pyrrobutamine, thenaldine, tolpropamine, triprolidine, bietanautin, bromdiphenhydramine, carbinoxamine, clemastine, diphenylpyraline, doxylamine, embramine, medrylamine, mephenhydramine, p-methyldiphenhydramine, orphenadrine, phenyltoloxamine, piprine hydrinate, setastine, alloclamide, chloropyramine, chlorothene, histapyrrodine, methafurylene, methaphenilene, methapyrilene, phenbenzamine, pyrilamine, talastine, thenyldiamine, thonzylamine, tripelennamihe, zolamine, cetirizine, chlorcyclizine, clocinizine., hydroxyzine, tricyclics,

antimigraine agents, hydrogenated ergot alkaloids, adrenoreceptor blockers, Ca antagonists, serotonin antagonists, platelet aggregation inhibitors, antidepressants such as, for example, alpiropride, dihydroergotamine, ergocornine, ergocorninine, ergocryptine, ergot, ergotamine, flumedroxone acetate, fonazine, methysergide, oxetorone, pizotyline, sumatriptan, anagrelide, argatroban, cilostazole, daltroban, defibrotide, enoxaparine, fraxiparine, indobufen, lamoparan, ozagrel, picotamide, plafibride, tedelparine, ticlopidine, triflusal,

bronchodilators such as, for example, ephedrine derivatives such as, for example, albuterol, bambuterol, bitolterol, carbuterol, clenbuterol, chlorprenaline, dioxethedrine, eprozinol, etafedrine, ethylnorepinephrine, fenoterol, hexoprenaline, isoetharine, isoproterenol, mabuterol, metaproterenol, N-methylephedrine, pirbuterol, procaterol, protokylol, reproterol, rimiterol, soterenol, terbutaline, tulobuterol, estrogens such as, for example, benzestrol, broparoestrol, chlorotrianisene, dienestrol, diethylstilbestrol, diethylstilbestrol dipropionate, dimestrol, fosfestrol, hexestrol, methallenestril, methestrol, colpormone, equilenin, equilin, conjugated estrogenic hormones, estrogen esters, estropipate, 17β-estradiol, estradiol, estradiol benzoate, estradiol 17β-cypionate, estriol, estrone, ethinylestradiol, mestranol, moxestrol, mytatrienediol, polyestradiol phosphate., quinestradiol, quinestrol,

gestagens such as, for example, allylestrenol, anagestone, chlormadinone acetate, delmadinone acetate, demegestone, desogestrel, dimethisterone, dydrogesterone, ethinylestrenol, ethisterone, ethynodiol, ethynodiol diacetate, flurogestone acetate, gestodene, gestonorone caproate, haloprogesterone, 17-hydroxy-16-methyleneprogesterone, 17α-hydroxyprogesterone, 17α-hydroxygesterone caproate, levonorgestrel, lynestrenol, medrogestone, medroxyprogesterone, megestrol acetate, melengestrol, norethindrone, norethindrone acetate, norethynodrel, norgesterone, norgestimate, norgestrel, norgestrienone, 19-norprogesterone, norvinisterone, pentagestrone, progesterone, promegestone, quingestrone, trengestone,

vasodilators such as, for example, bencyclan, ciclonicate, cinnarizine, citicoline, diisopropylamine dichloroacetate, eburnamonine, fenoxedil, ibudilast, ifenprodil, nafronyl, nicametate, nicergoline, ninodipine, papaverine, pentifylline, tinofedrine, vincamine, vinpocetine, amotriphene, bendazole, benfurodil hemisuccinate, benziodarone, chloracyzine, chromonar, clobenfurol, clonitrate, dilazep, dipyridamol, dropenilamine, efloxate, erythritol, erythrityl tetranitrate, etafenone, floredil, ganglefen, hexestrol bis(β-diethylaminoethyl ether), hexobendine, isosorbide dinitrate, itramine tosylate, khellin, lidoflazine, mannitol hexanitrate, medibazine, nicorandil, nitroglycerine, pentaerythritol tetranitrate, pentrinitrol, pimethylline, prenylamine, propatyl nitrate, pyridofylline, trapidil, tricromyl, trimetazidine, troInitrate phosphate, visnadine, bamethane, betahistine, bradykinin, brovincamine, bufoniod, buflomedil, butalamine, cetiedil, ciclonicate, cinepazide, cyclandelate, eledoisine, hepronicate, inositol niacinate, isoxsuprine, kallidine, kallikrein, moxisylyt, nicofuranose, nylidrine, piribedil, suloctidil, xanthinal and niacinate, and also nicotine.

### EXAMPLE

Four transdermal therapeutic systems (formulations nos. 1 to 4) were prepared, which as active compounds contained 17β-estradiol or testosterone in the polymer matrix. As a back layer, a polyethylene terephthalate film was used; the area weight of the polymer matrix was 100 g/m². (The thickness of the polymer matrix can preferably be between 15 to 30 μm.)

For comparison of the behavior with respect to the active compound utilization, on the one hand Durotak 2287 was employed for the polymer matrix as a contact-adhesive polyacrylate having a functional group, while in the transdermal therapeutic systems according to the invention the adhesive GMS 3083 from Solutia was used as a contact-adhesive polyacrylate without a functional group within the meaning of this description.

Tables 1 and 2 show the cumulated amounts of active compound for the respective polymer matrices which were measured in a Franz's cell which was equipped with an EVA membrane.

TABLE 1
Release behavior from estradiol-containing
polymer matrices

| | | | Cumulated amount of |
|---|---|---|---|
| | | | active compound in |
| No. | Ingredients | % content | [μg/cm²] (after 3 days) |
| 1 | 17β-estradiol | 1.00 | 170.3 |
| | Durotak 2287 | 99.00 | |
| 2 | 17β-estradiol | 1.00 | 240.6 |
| | GMS 3083 | 99.00 | |

TABLE 2
Release from testosterone-containing polymer
matrices

| | | | Cumulated amount of |
|---|---|---|---|
| | | | active compound in |
| No. | Ingredients | % content | [μg/cm²] (after 3 days) |
| 1 | testosterone | 2.00 | 575.3 |
| | Durotak 2287 | 98.00 | |
| 2 | testosterone | 2.00 | 675.5 |
| | GMS 3083 | 98.00 | |

The active compound utilization results from the cumulated amount of active compound divided by the amount of active compound contained in the TTS.

As is seen, in the case of estradiol it was possible by use of the polyacrylate adhesive without functional groups to achieve a 40% better active compound utilization, in the case of testosterone a 17% better active compound utilization. In other words: The cumulative flux is higher by the factor 40% and 17% respectively. Thus with the same active compound loading a better active compound utilization can be achieved.

1. A transdermal therapeutic system comprising back layer, protective layer and at least one active-compound-containing polymer matrix comprising a polyacrylate, the polyacrylate being a homopolymer, copolymer or block copolymer which can be prepared by polymerization of a monomer mixture consisting of:
a) a monomer or a mixture of monomers from the group consisting of the esters of acrylic or methacrylic acid, which carry linear, branched and/or cyclic aliphatic C₁-C₁₂ substituents without another functional group,
b) acrylic acid, methacrylic acid, 2-hydroxyethyl acrylate, 2-hydroxyethyl methacrylate, 3-hydroxypropyl acrylate and/or 3-hydroxypropyl methacrylate, the content of these monomers in the total being below 2% by weight,
c) vinyl acetate in a content of below 20% by weight,
d) crosslinkers in a content of below 0.5% by weight
e) auxiliaries from the group consisting of the antioxidants, stabilizers and/or alkylmercaptans in a content of below 0.1% by weight.

2. The transdermal therapeutic system as claimed in claim 1, characterized in that the polymerization of the monomer mixture is carried out by ionic, free-radical or light-induced means, in water which can contain emulsifiers or in organic solvents as a reaction medium.

3. The transdermal therapeutic system as claimed in claim 1 or 2, characterized in that the monomer mixture contains vinyl acetate in a content of below 5% by weight.

4. The transdermal therapeutic system as claimed in one or more of claims 1 to 3, characterized in that aluminum acetylacetonate, allyl glycidyl ether and/or glycidyl methacrylate is used in the monomer mixture as a crosslinker.

5. The transdermal therapeutic system as claimed in one or more of claims 1 to 4, characterized in that the group of esters of acrylic or methacrylic acid which contain linear, branched and/or cyclic aliphatic C₁-C₁₂ substituents without another functional group consists of n-butyl acrylate, n-butyl methacrylate, ethyl acrylate, 2-ethylhexyl acrylate, ethyl methacrylate, methyl acrylate, methyl methacrylate, tert-butyl acrylate, sec-butyl acrylate, tert-butyl methacrylate, cyclohexyl methacrylate, 2-ethylhexyl methacrylate, isobornyl methacrylate, isobutyl methacrylate, isopropyl acrylate, isopropyl methacrylate and mixtures of these monomers.

6. The transdermal therapeutic system as claimed in one or more of claims 1 to 5, characterized in that the monomer mixture is free of acrylic acid, methacrylic acid, 2-hydroxyethyl acrylate, 2-hydroxyethyl methacrylate, 3-hydroxypropyl acrylate and/or 3-hydroxypropyl methacrylate.

7. The transdermal therapeutic system as claimed in one or more of claims 1 to 6, characterized in that the active compound is a pharmaceutically active substance from the group consisting of the α-adrenoreceptor agonists, the β-adrenoreceptor agonists, the α-adrenoreceptor blockers, the β-adrenoreceptor blockers, the analgesics (narcotics), the analgesics (non-narcotics), the androgens, the anesthetics, the antiallergics, the antiandrogens, the antianginals, the antiarrhythmics, the penicillins, the antidiabetics, the antihistaminics, the antimigraine agents, the hydrogenated ergot alkaloids, the Ca antagonists, the serotonin antagonists, the platelet aggregation inhibitors, the anti-depressants, the bronchodilators, the estrogens, the gestagens, the vasodilators and nicotine.

8. The transdermal therapeutic system as claimed in one or more of claims 1 to 7, characterized in that the active compound is a hormone or a combination of hormones.

9. The transdermal therapeutic system as claimed in claim 8, characterized in that the hormone is 17β-estradiol, ethinylestradiol, estradiol acetate, levonorgestrel, norethindrone, norethindrone acetate or testosterone.

10. The use of a transdermal therapeutic system as claimed in one of claims 1 to 9 for the treatment of hypogonadism, for hormone substitution therapy or for contraception.
